(19) United States
(12) Patent Application Publication
(10) Pub. No.: US 2012/0095325 A1
(43) Pub. Date: Apr. 19, 2012

(54) TREATMENT OF BRAIN DISEASES VIA ULTRASOUND/MAGNETIC TARGETING DELIVERY AND TRACING OF THERAPEUTIC AGENTS

(75) Inventors: Kuo-Chen Wei, Taoyuan County (TW); Hao-Li Liu, Taoyuan County (TW); Mu-Yi Hua, Taoyuan County (TW)

(73) Assignee: Chang Gung Medical Foundation, Linkou Branch, Gueishan Township (TW)

(21) Appl. No.: 13/273,368

(22) Filed: Oct. 14, 2011

Related U.S. Application Data
(60) Provisional application No. 61/393,518, filed on Oct. 15, 2010.

(51) Int. Cl. A61B 5/055; A61N 7/00; B82Y 5/00

(52) U.S. Cl. 600/411; 977/904

(57) ABSTRACT

Disclosed herein is a method for treating a brain disease in which focused ultrasound and magnetic targeting are applied to a subject in need of such treatment, so that therapeutic agent-magnetic nanoparticle composites are directed across the blood-brain barrier to a designated locus inside the brain of the subject. Each of the composites includes a magnetic nanoparticle that is formed of an iron-based core and a shell encapsulating the iron-based core, and a therapeutic agent that is bound to the shell of the magnetic nanoparticle. The magnetic nanoparticle has a size ranging from 5 to 200 nm. The iron-based core has a crystalline structure that imparts the composites with a sufficiently high magnetization, thereby enhancing magnetic targeting of the composites to the designated locus inside the brain of the subject. The magnetic targeting treatment is conducted via a magnet providing a magnetic flux density not less than 0.18 T.

### CROSS-REFERENCE TO RELATED APPLICATIONS

This application claims priority of U.S. provisional application No. 61/393,518, filed on Oct. 15, 2010.

### BACKGROUND OF THE INVENTION

1. Field of the Invention

This invention relates to a method for treating a brain disease in which focused ultrasound sonication and a magnetic targeting treatment are applied to a subject in need of such treatment, so that magnetic nanoparticles carrying a macromolecular therapeutic agent are directed to cross the blood-brain barrier (BBB) of said subject and target a selected locus inside the brain of said subject. The method also employs medical imaging to trace the magnetic nanoparticles carrying the macromolecular therapeutic agent.

2. Description of the Related Art

The blood-brain barrier (BBB) in the central nervous system (CNS) excludes molecules larger than 400 Da to enter the brain parenchyma, thereby protecting the brain parenchyma from being damaged by toxic foreign substances (W. M. Pardridge (2002), Neuron, 36:555-558). However, BBB also prohibits delivery of many potentially effective diagnostic or therapeutic agents and restricts the enhanced permeability and retention (EPR) of therapeutic nanoparticles. Many factors affect EPR, including the pH, polarity, and size of the delivered substance. Even when pathologic processes compromise the integrity or function of the BBB, EPR can be limited by microenvironmental characteristics such as hypovascularity, fibrosis, or necrosis (D. Begley and M. W. Brightman (2003), Peptide Transport and Delivery into the Central Nervous System, eds. L. Prokai, K. Prokai-Tatrai (Birkhäuser Verlag, Basel), pp. 39-78; J. Kreuter (2001), Adv. Drug Deliv. Rev., 47:65-81; PR Lockman et al. (2002), Drug Dev. Ind. Pharm., 28:1-13).

U.S. Pat. No. 5,752,515 discloses image-guide methods and apparatus for ultrasound delivery of compounds (e.g., a neuropharmaceutical), through the blood-brain barrier to selected locations in the brain.

U.S. Pat. No. 6,514,221 discloses a method of opening a blood-organ barrier of a subject, which includes providing an exogenous agent configured to facilitate opening of the blood-organ barrier, administering the exogenous agent to a desired region of the subject, and applying energy to the desired region of the subject while the exogenous agent is present in the desired region, the energy being in a blood-organ-barrier-opening amount sufficient to induce opening of the blood-organ barrier of the subject with the exogenous agent present and below a damage amount sufficient to induce thermal damage to tissue in the absence of the exogenous agent. The exogenous agent contains preformed gaseous bubbles. The energy applied is ultrasound energy and the exogenous agent contains at least one of a high concentration of gas, solid particles configured to vaporize in response to body temperature, solid particles configured to vaporize in response to the ultrasound energy, liquid configured to vaporize in response to body temperature, liquid configured to vaporize in response to the ultrasound energy, micro particles configured to act as cavitation sites, solid particles having higher acoustic impedance than tissue in the desired region, and liquid with a high ultrasound absorption coefficient.

US 20090005711 A1 discloses a system and method for opening the blood-brain barrier in the brain of a subject.

US 20100143241 A1 discloses a method for opening the blood-brain barrier (BBB) using ultrasound and preformed microbubbles.

The disclosures of the aforesaid US patent documents are incorporated herein by reference in their entirety.

In the presence of microbubbles and with use of a low-energy burst tone, focused ultrasound (FUS) can increase the permeability of the BBB (K. Hynynen et al. (2006), J. Neurosurg., 105:445-454). This noninvasive procedure disrupts the BBB locally rather than systemically, minimizing off-target effects. Furthermore, the disruption is reversible within several hours, providing a window of opportunity to achieve local delivery of chemotherapeutic agents in brains with intact or compromised BBBs. However, drug delivery by such approach is passive, relying on the free diffusion of the therapeutic agents across BBB. In addition, the conventional FUS-induced BBB opening procedure is only available to deliver small-sized therapeutic agents.

Advances in nanotechnology and molecular biology have allowed development of novel nanomedical platforms (O. C. Farokhzad and R. Langer (2006), Adv. Drug Deliv. Rev., 58:1456-1459; N. Sanvicens and M. P. Marco (2008), Trends Biotechnol., 26:425-433; 0. Veiseh et al. (2010), Adv. Drug Deliv. Rev., 62:284-304). Such approaches allow simultaneous diagnostic imaging and drug delivery monitoring in vivo in real time (C. Sun et al. Adv. Drug Deliv. Rev., 60:1252-1265; V. P. Torchilin (2006), Adv. Drug Deliv. Rev., 1532-1555). Magnetic nanoparticles (MNPs) have intrinsic magnetic properties that enable their use as contrast agents in magnetic resonance imaging (MRI)(O. Veiseh et al. (2010), supra; C. Zimmer et al. (1997), Exp. Neurol., 143:61-69). Because MNPs are also sensitive to external magnetic forces, magnetic targeting (MT) actively enhances their deposition at the target site, increasing the therapeutic dose delivered beyond that obtainable by passive diffusion (B. Chertok et al. (2007), J. Control Release, 122:315-323).

The superparamagnetic properties of MNPs allow them to be guided by an externally positioned magnet and also provide contrast for MRI. However, their therapeutic use in treating brain diseases in vivo is limited by insufficient local accumulation and retention resulting from their inability to traverse biological barriers, in particular BBB.

The applicants attempted to develop a new approach for the treatment of brain diseases, in which FUS sonication and magnetic targeting (MT) are combined to deliver therapeutic MNPs into brains under concurrent MRI monitoring. FUS sonication creates the opportunity to deliver therapeutic MNPs by passive local EPR, while externally applied magnetic forces actively increase the local MNP concentration. The applicants surprisingly found that the combination of FUS sonication and MT permitted the delivery of large molecules into the brain. Furthermore, the deposition of the therapeutic MNPs can be monitored and quantified in vivo by MRI.

### SUMMARY OF THE INVENTION

Therefore, this invention provides a method for treating a brain disease, comprising:
(i) delivering therapeutic agent-magnetic nanoparticle composites to the vicinity of the blood-brain barrier of a subject in need of such treatment;
(ii) applying focused ultrasound to the subject so as to open the blood-brain barrier of the subject;
(iii) applying a magnetic field to the subject to direct the therapeutic agent-magnetic nanoparticle composites across the blood-brain barrier to a designated locus inside the brain of the subject;
(iv) monitoring the quantity of the therapeutic agent-magnetic nanoparticle composites present at the designated locus by magnetic resonance imaging;
and optionally, directing more therapeutic agent-magnetic nanoparticle composites to the designated locus inside the brain of the subject by repeating steps (i) to (iv),
wherein each of the therapeutic agent-magnetic nanoparticle composites is constructed to comprise:
(a) a magnetic nanoparticle formed of an iron-based core and a shell encapsulating the iron-based core, the shell comprising a biological compatible polymer, and
(b) a therapeutic agent bound to the shell of the magnetic nanoparticle;
wherein the magnetic nanoparticle has an average particle size ranging from 5 to 200 nm and wherein reaction conditions used in preparation of the iron-based core of the magnetic nanoparticle are controlled, so that the iron-based core has a crystalline structure that imparts the magnetic nanoparticle composites with a sufficiently high magnetization, thereby enhancing magnetic targeting of the therapeutic agent-magnetic nanoparticle composites to the designated locus inside the brain of the subject; and
wherein in step (iii), the magnetic field is generated by a magnet that provides a magnetic flux density not less than 0.18 T.

### BRIEF DESCRIPTION OF THE DRAWINGS

The above and other objects, features and advantages of this invention will become apparent with reference to the following detailed description and the preferred embodiments taken in conjunction with the accompanying drawings, in which:

FIG. 1 shows the magnetic attraction effect of an externally applied magnetic field upon a commercially available carboxydextran-coated Resovist® (Schering) and MNP-3 prepared according to this invention under different medium viscosities, in which W: distilled water, 37° C., viscosity of 0.7 mPa·s; and S, bovine serum, 37° C., viscosity of 1.35 mPa·s.

FIG. 2 shows that epirubicin was immobilized on epirubicin-MNP-3 composite prepared according to this invention, as verified by phase (Upper) and fluorescence (Lower) confocal microscopy (scale bar: 10 μm).

FIG. 3 shows the FT-IR spectra of epirubicin, MNP-3, and the epirubicin-MNP-3 composite;

FIG. 4 shows the quantification of epirubicin immobilized on 1 mg of MNP-3 versus added epirubicin by HPLC, in which the experimental data were expressed as mean±SD (n=6);

FIG. 5 shows the cytotoxic effects of MNPs, epirubicin and epirubicin-MNPs in C6 cells, in which panel A, viability of C6 cells in the presence of different concentrations of drug-free MNPs; and panel B, viability of C6 cells after incubation with free epirubicin, epirubicin-MNPs and epirubicin-MNPs plus magnet targeting (subjected to an 800-G magnetic field). Cell viability was determined by XTT assay. The experimental data were expressed as mean±SD (n=8);

FIG. 6 shows the effect of magnetic flux density on magnet targeting (MT), in which panel A, T2-weighted MRI (upper) and the corresponding R2 maps (lower) of brains; and panel B, percent increase of relaxivity against the contralateral brain hemisphere. Three magnets with peak flux densities of 0.18, 0.4, and 0.55 T were tested. For all experiments, MNP-3 was used and the MT time was fixed at 6 hr. The experimental data were expressed as mean±SD (n=3);

FIG. 7 shows the in vivo imaging of MNP distribution in the brain (Top, T2-weighted images; Middle, T2*-weighted images; Bottom, combined R2 maps and T2-weighted images), in which panel A, FUS sonication and MNP injection; panel B, FUS followed by MT for 3 hrs after MNP injection; and panel C, FUS followed by MT for 6 hrs after MNP injection;

FIG. 8 shows the measurement of epirubicin accumulation in experimental animals over time, in which panel A, epirubicin concentration (ng of epirubicin/g tissue) in the experimental brain hemisphere; and panel B, epirubicin concentration (ng of epirubicin/g tissue) in the contralateral brain hemisphere. MT-only means MT with MNP-3 (i.e., without FUS); FUS-only means FUS with MNP-3 (i.e., without MT); and MNP-1, MNP-2 and MNP-3 indicate MNPs administered in conjunction with combined FUS and MT; and panel C, Correlation between epirubicin concentration and the ΔR2 values (i.e., R2 values after subtraction of baseline values) as measured by MRI. The experimental data were expressed as mean±SD (n=3);

FIG. 9 shows the in vivo T2-weighted MRI (left side) and the corresponding R2 maps (right side) of brain tumors without (panel A) or with (panel B) FUS and MT, and the measured relaxivities in tumor regions from the control and experimental groups (panel C). The experimental data were expressed as mean±SD (n=3);

FIG. 10 shows three TEM images of brain tumors, in which the presence of MNPs inside opened tight junction structures (TJ) and uptake by tumors cells (TC) and macrophages (M) are indicated. Numerous caveolae in tumor cells or macrophages indicate apoptosis resulting from the uptake of epirubicin-MNPs. EC means endothelial cell;

FIG. 11 shows the confocal micrographs of tissue from tumor (panel A) and contralateral brain regions (panel B). Dark structures in the phase micrographs show MNPs (left side); fused fluorescence images (right side) indicate the presence of epirubicin (red) and DAPI stained nuclei (blue). Arrows indicate the capillaries; epirubicin occurs in the capillary beds but does not penetrate into the brain parenchyma;

FIG. 12 shows the histological examination of treated tumor (T) and contralateral brain (C) regions, in which panels a and f (H&E staining, HE), panels b and g (Prussian blue staining, PB) and panels c and h (fluorescence images) show epirubicin distribution (red); panels d and I (DAPI-stained fluorescent images) shows nucleus distribution (blue); and panels e and j show fused fluorescence images of epirubicin and DAPI-stained cells; and

FIG. 13 shows the percent increase in tumor volume measured from 1 to 8 days after different treatments (epirubicin-MNP-3 only, epirubicin-MNP-3+FUS, and epirubicin-MNP-3+FUS/MT) (panel A) and the Kaplan-Meier survival plots of the animal experiments (panel B), in which Epirubicin-MNP delivery combined with FUS/MT provided the most significant suppression of tumor progression (P=0.0348) and survival (P=0.0002) relative to untreated controls.

### DETAILED DESCRIPTION OF THE INVENTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of the present invention. Indeed, the present invention is in no way limited to the methods and materials described.

The applicants have attempted to develop a method for the treatment of brain diseases by delivering therapeutic agents, in particular those having a molecular weight greater than 400 Dalton, to a designated location inside the brain.

The applicants surprisingly found that the combined use of focused ultrasound and magnetic targeting synergistically delivered therapeutic MNPs across the blood-brain barrier to enter the brain both passively and actively. Therapeutic MNPs were characterized and evaluated both in vitro and in vivo, and MRI was used to monitor and quantify their distribution in vivo. This technique could be used in normal brains or in those with tumors, and significantly increased the deposition of therapeutic MNPs in brains with intact or compromised blood-brain barriers. The applicants contemplate that synergistic targeting and image monitoring are powerful techniques for the delivery of macromolecular chemotherapeutic agents into the CNS under the guidance of MRI.

Accordingly, this invention provides a method for treating a brain disease, comprising:
(i) delivering therapeutic agent-magnetic nanoparticle composites to the vicinity of the blood-brain barrier of a subject in need of such treatment;
(ii) applying focused ultrasound to the subject so as to open the blood-brain barrier of the subject;
(iii) applying a magnetic field to the subject to direct the therapeutic agent-magnetic nanoparticle composites across the blood-brain barrier to a designated locus inside the brain of the subject;
(iv) monitoring the quantity of the therapeutic agent-magnetic nanoparticle composites present at the designated locus by magnetic resonance imaging;
and optionally, directing more therapeutic agent-magnetic nanoparticle composites to the designated locus inside the brain of the subject by repeating steps (i) to (iv),
wherein each of the therapeutic agent-magnetic nanoparticle composites is constructed to comprise:
(a) a magnetic nanoparticle formed of an iron-based core and a shell encapsulating the iron-based core, the shell comprising a biological compatible polymer, and
(b) a therapeutic agent bound to the shell of the magnetic nanoparticle;
wherein the magnetic nanoparticle has an average particle size ranging from 5 to 200 nm and wherein reaction conditions used in preparation of the iron-based core of the magnetic nanoparticle are controlled, so that the iron-based core has a crystalline structure that imparts the magnetic nanoparticle composites with a sufficiently high magnetization, thereby enhancing magnetic targeting of the therapeutic agent-magnetic nanoparticle composites to the designated locus inside the brain of the subject; and
wherein in step (iii), the magnetic field is generated by a magnet that provides a magnetic flux density not less than 0.18 T.

The method of this invention synergistically combines FUS and MT to increase therapeutic agent delivery to the brains using a “safe” level of FUS exposure, and the therapeutic agent can quantitatively accumulate at the designated locus inside the brain of the subject.

According to this invention, step (ii) may be performed by applying to the subject a planar/focused ultrasound beam having a frequency ranging from 20 kHz to 10 MHz, at a sonication duration ranging from 100 nanoseconds to 30 minutes, with continuous wave or burst mode operation, in which frequency of burst mode repetition varies from 0.01 Hz to 1 MHz.

According to this invention, prior to step (ii), the subject is administered with ultrasound microbubbles that enhance focused ultrasound.

The method of this invention is able to deliver molecules larger than 400 Da, in particular molecules larger than 1000 kDa across the blood-brain barrier since the method of this invention employs the magnetic nanoparticle that has sufficient saturated magnetization, and the magnetic field that has a sufficient strength.

According to this invention, in step (iii), the magnetic field is generated by a magnet that provides a magnetic flux density ranging from 0.18 T to 0.55 T. In a preferred embodiment of this invention, in step (iii), the magnetic field is generated by a magnet that provides a magnetic flux density of 0.55 T.

In addition, the magnetic nanoparticles of this invention exhibit dual functions, i.e., a pharmaceutical carrier for the therapeutic agent and an imaging probe for MRI monitoring.

According to this invention, the magnetic nanoparticle has a saturated magnetization ranging from 32.6 emu to 81.7 emu based on one gram of the magnetic nanoparticle. Preferably, the magnetic nanoparticle has a saturated magnetization greater than 70 emu per gram of the magnetic nanoparticle. In a preferred embodiment of this invention, the magnetic nanoparticle has a saturated magnetization of 81.7 emu based on one gram of the magnetic nanoparticle.

According to this invention, the iron-based core of the magnetic nanoparticle has a relaxivity not less than 30 mM⁻¹s⁻¹. Preferably, the iron-based core of the magnetic nanoparticle has a relaxivity in a range from 30 to 400 mM⁻¹s⁻¹. More preferably, the iron-based core of the magnetic nanoparticle has a relaxivity in a range from 30 to 217 mM⁻¹s⁻¹. In a preferred embodiment of this invention, the iron-based core of the magnetic nanoparticle has a relaxivity>100 mM⁻¹s⁻¹.

According to this invention, the iron-based core of the magnetic nanoparticle is made of a material selected from the group consisting of Fe₂O₃ and Fe₃O₄. In a preferred embodiment of this invention, the iron-based core of the magnetic nanoparticle is made of Fe₃O₄.

According to this invention, the biological compatible polymer used to form the shell of the magnetic nanoparticle is selected from the group consisting of polyaniline, polylactic acid (PLA), polyglycolic acid (PGA), polylactic polyglycolic acid (PLGA), dextran, dextran grafted with poly(N-isopropylacrylamide-co-N,N-dimethylacrylamide), dextran grafted with poly(phosphoester urethane), polycaprolactone (PCL), polyhydroxybutyrate (PHB), polyethylene glycol (PEG)-modified PLGA, and poly(L-lysine)-g-polyethylene glycol) (PLLgPEG)-modified PLGA.

In a preferred embodiment of this invention, the biological compatible polymer is carboxy-functionalized polyaniline. In a more preferred embodiment of this invention, the biological compatible polymer is poly[aniline-co-N-(1-one-butyric acid)]aniline.

The method of this invention may be used to treat a brain disease selected from tumors, cancer, degenerative disorders, sensory and motor abnormalities, seizure, infection, immunologic disorder, mental disorder, behavioral disorder, localized CNS disease, and combinations thereof. In a preferred embodiment of this invention, the brain disease is a brain cancer, in particular glioma.

According to this invention, the therapeutic agent includes, by way of non-limiting examples, any of neuropharmacologic agents, neuroactive peptides (e.g., hormones, gastrointestinal peptides, angiotensin, sleep peptides, etc.), proteins (e.g, calcium binding proteins), enzymes (e.g., cholineacetyltransferase, glutamic acid decarboxylase, etc.), gene therapy agents, neuroprotective or growth factors, biogenic amines (e.g., dopamine, GABA), trophic factors to brain or spinal transplants, immunoreactive proteins (e.g., antibodies to neurons, myelin, antireceptor antibodies), receptor binding proteins (e.g., opiate receptors), radioactive agents (e.g., radioactive isotopes), antibodies, and cytotoxins, among others.

When the method is used to treat a brain cancer such as glioma, the therapeutic agent may be an anti-brain cancer drug selected from the group consisting of epirubicin, doxorubicin, 1,3-bis(2-chloroethyl)-1-nitrosourea (BCNU), N(2-chloroethyl)-N′-cyclohexyl-N-nitrosourea (CCNU), methyl 6-(3-(2-chloroethyl)-3-nitrosoureido (MCNU), (2-chloroethyl)nitrosourea (CI-ENU), N-(2-hydroxyethyl)-N-nitrosourea (HO-ENU), and 1-methyl-1-nitrosourea (MNU).

The quantity of the therapeutic agent-magnetic nanoparticle composites can be monitored by magnetic resonance imaging since the magnetic nanoparticles have intrinsic magnetic properties. Consequently, the amount of the therapeutic agents delivered to the brain of the subject can be controlled so as to effectively treat the brain disease.

This invention will be further described by way of the following examples. However, it should be understood that the following examples are solely intended for the purpose of illustration and should not be construed as limiting the invention in practice.

### EXAMPLES

### 1. Preparation of Magnetic Nanoparticles (MNPs)

Three different MNPs used in the study of this invention, namely MNP-1, MNP-2 and MNP-3, were prepared as follows:

First of all, three kinds of Fe₃O₄ cores, namely Core-1, Core-2 and Core-3, were prepared separately by different coprecipitation reactions. Briefly, FeCl₃ (4.32 mmol) and FeCl₂.4H₂O (2.16 mmol for Core-1, and 6.48 mmol for Core-2 and Core-3) were dissolved in 400 mL of deionized water and the resultant three solutions were stirred for 5 min under N₂ gas, followed by heating slowly to 50° C. (for Core-3) or 60° C. (for Core-1 and Core-2). Thereafter, 20 mL of 0.864 N NaOH was added into each solution over a 5-min (for Core-1 and Core-2) or 60-min (for Core-3) period, after which the temperature was increased to 80° C. for 5 min (for Core-1 and Core-2) or 15 min (for Core-3). After Fe₃O₄ formation, the three solutions were quenched rapidly in an ice water bath, followed by sonication at 300 W for 1 h so as to uniformly disperse the Fe₃O₄ cores formed therein. Each of the three solutions was poured into a separation funnel and the Fe₃O₄ cores formed therein were attracted onto the inner wall of the separation funnel by a strong magnet. Deionized water was continuously poured into the separation funnel to wash the attracted Fe₃O₄ cores until the washed solution became colorless and neutral.

Poly[aniline-co-sodium N-(1-one-butyric acid) aniline] (SPAnNa) having carboxyl groups was synthesized using supercritical carbon dioxide as a reaction medium. Briefly, succinic anhydride (0.83 g, 8.3 mmole) and 0.55 g (4.15 mmole) of AlCl₃ were dissolved in 10 mL of 1-methyl-2-pyrrolidone (NMP), respectively, and the AlCl₃ solution was added slowly into the succinic anhydride solution under nitrogen gas, followed by heating to 50° C. for 2 hrs. Polyaniline (0.3 g, 0.83 mmole) was dissolved in 50 mL of NMP and then mixed with the AlCl₃—succinic anhydride solution. The resultant mixture was placed in a supercritical reactor and heated to 50° C. in a water bath. CO₂ was introduced into the reactor and compressed to 2400 psig using a high-pressure pump. In supercritical CO₂, it takes 4 hours for the nuclear affinity substitution reaction to occur. After reaction, the pressure within the reactor was released to standard atmospheric levels, and the reaction product was mixed with 150 mL of 0.5 M HCl and was then stirred for 1 hr, thereby inducing the aggregation to purify the products. The resultant green precipitate was filtered and washed with deionized water until the filtered solution became neutral. 200 mL of 0.5 M NaOH was added to the precipitate and stirred for 36 hrs until the de-doped solution (SPAnNa) turned dark blue to become sodium type for increasing the solubility in aqueous solution. After filtration to remove the precipitate (low immobile rate), the solution was refiltered using a Spectra/Por 3 membrane with a molecular weight cutoff of 3500 and purified using deionized water to eliminate the product with low molecular weight. This purification procedure separated the aqueous NaOH from the solution, neutralizing SPAnNa inside the membrane and producing a deep blue solution of poly[aniline-co-sodium N-(1-one-butyric acid)]aniline (SPAnNa). A deep green solution of poly[aniline-co-N-(1-one-butyric acid)]aniline (SPAnH) can be formed using a H⁺-type cation exchange resin to replace the Na⁺ of SPAnNa with H⁺.

1.5 mL of SPAnNa (5.5 mg/mL) was uniformly mixed with 5 mL of each of cores 1, 2 and 3 prepared above (10 mg/mL), and the resultant mixture was doped slowly by addition of 0.2 M HCl. Acid doping of SPAnNa induces the formation and aggregation of SPAnH. Cores 1, 2 and 3 were therefore encapsulated in a SPAnH shell to form MNP-1, MNP-2 and MNP-3, respectively, each being separated from the solution using a strong magnet, followed by washing with deionized water and continuous sonication until the washed solution became neutral.

### 2. Characterization of MNP

The three kinds of MNPs as prepared above were dispersed in deionized water and analyzed by Fourier transform infrared (FT-IR) spectroscopy, superconducting quantum interference, dynamic light scattering, X-ray diffraction and transmission electron microscopy (TEM).

A drop of a diluted MNP suspension was deposited on a 300 mesh silicon-monoxide support film and dried under vacuum for 2 hrs. Images were acquired on a Phillips 400 transmission electron microscope operating at 100 kV.

The superparamagnetic properties and magnetization of MNPs were measured using a superconducting quantum interference device (MPMS-7; Quantum Design).

FT-IR spectra were acquired using a TENSOR 27 FT-IR spectrometer (Bruker) with a resolution of 4 cm. MNP samples were milled with KBr and pressed into a pellet for X-ray diffraction pattern analysis. Patterns were acquired from lyophilized samples with a D5005 X-ray diffractometer (Siemens) using Cu—Kα radiation (λ of 1.541 Å) at 40 kV and 40 mA. Zeta potentials and hydrodynamic sizes of MNPs were measured in water using a dynamic light scattering particle size analyzer (ZEN3600; Malvern).

The polymers covering the MNPs were quantified by inductively coupled plasma-optical emission spectrometry (ICP-OES) using a Varian 720-ES spectrometer. The relaxivities of the three MNPs generated for the study of this invention, as well as that of a commercially available carboxydextran-coated Resovist® (60-nm hydrodynamic size; Schering), were measured in vitro in gel phantoms and in vivo. Standard samples of poly[aniline-co-sodium N-(1-one-butyric acid) aniline]-coated MNPs (0-6.48 mmol/kg iron) were prepared as gel phantoms (1% gelatin) in 24-well plates. Standard R2 measurements were performed using a 3-T magnetic resonance imager (Trio with Tim, Magnetom; Siemens). The relaxivities reported are the mean values of five measurements.

### Results:

In the study of this invention, the polymer poly[aniline-co-N-(1-one-butyric acid)]aniline (SPAnH) was used to encapsulate the Fe₃O₄ core. This process decreases the aggregation typical of MNPs and improves their stability in aqueous solutions. The FT-IR spectra of MNP-1, MNP-2 and MNP-3 indicate that the surface of each of cores 1, 2 and 3 was covered with a layer of the SPAnH polymer and that the outermost layer of each synthesized MNP maintained the —NH and —COOH groups, which could be used to immobilize drugs or other biomaterials (data not shown).

The physical properties, in terms of mean hydrodynamic size and particle size, saturated magnetization and spin-spin relaxation rate (R2), of the commercially available MNP Resovist® and the three MNPs prepared in this invention are summarized in Table 1.

TABLE 1
Physical properties of the MNPs prepared
in this invention and Resovist ®.

| | | TEM | Saturated | |
|---|---|---|---|---|
| | Hydrodynamic | diameter | magnetization | R2 |
| | diameter (nm) | (nm) | (emu/g MNP) | (mM⁻¹s⁻¹) |
| Resovist ® | 63.8 | 5.9 | 73.7 | 98.4 |
| MNP-1 | 73.7 | 10.9 | 51.8 | 30.0 |
| MNP-2 | 75.8 | 11.4 | 65.9 | 102.3 |
| MNP-3 | 83.4 | 12.3 | 81.7 | 185.0 |

As measured by TEM, MNP-3 has a mean diameter of 12.3 nm. This is significantly smaller than the hydrodynamic sizes measured by dynamic light scattering (64 nm for Resovist®, 74-83 nm for MNPs-1-3), although such differences could be attributable to solvent effects.

Magnetization of MNPs is crucial for their utility in magnetic targeting, and crystallinity significantly affects this parameter. During synthesis, the crystallinity of the MNPs was manipulated by controlling the reaction conditions. Referring to Table 1, amongst the four kinds of MNPs tested, MNP-3 has the highest degree of saturated magnetization. MNP-3 was also found to exhibit the best crystallinity (data not shown).

Administration of MNPs into biological tissues profoundly alters the spin-spin relaxation rate (R2), which can thus serve as an indicator of a MRI contrast agent. Referring to Table 1, the R2 value, and hence the detection sensitivity, of MNP-3 is highest amongst the four kinds of MNPs tested. MNP-3 is expected to be most susceptible to magnetic targeting.

The measured zeta potentials of MNP-1, MNP-2 and MNP-3 are similar to that of Resovist® (approximately 45 mV).

### 3. Magnetic Targeting Efficacy of MNPs In Vitro

A thin tubing (0.25-mm internal diameter) was positioned 2 or 4 mm below the pole of a 0.55-T magnet. An MNP suspension (either Resovist® or MNP-3, 2.5 mg iron/mL) was infused continuously using a syringe pump through one end of the tubing and collected at the other end at a fixed flow rate of 15.0 cm/s. Serial images of the tubing segment located near the pole were acquired with a digital camera before and after initiation of the magnetic field at 2-min intervals over a 10-min period. To determine the effect of viscous drag on magnetic targeting, the accumulation of Resovist® or MNP-3 was tested in 37° C. distilled water (viscosity, 0.7 mPa·s) and 37° C. bovine serum (viscosity, 1.35 mPa·s). Viscosity was measured using a V90000 viscometer (Fungilab). The amount of magnetic nanoparticles attracted by the external magnetic field in a test medium was calculated by inductively coupled plasma optical emission spectrometry (ICP-OES).

### Results:

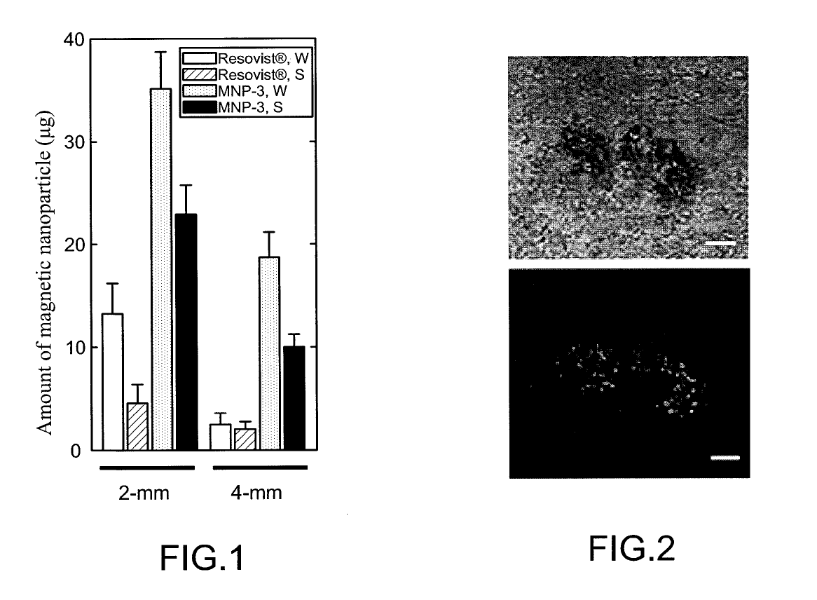

The ability of MNPs to be attracted by an external magnetic field was tested in vitro. Referring to FIG. 1, Resovist® failed to aggregate when it was infused through plastic tubing at a constant flow rate and at a fixed distance from an external applied magnetic field. In contrast, under the same conditions, significant accumulation of MNP-3 was observed. An approximate doubling of the medium viscosity led to a nearly 50% decrease in MNP aggregation, suggesting that the efficiency of magnetic targeting is inversely proportional to viscous drag. Based on the obtained results, MNP-3 is expected to have a great ability to be magnetically targeted to a desired location in a living subject.

### 4. Preparation of Epirubicin-MNP Composite

24 mg of 1-ethyl-3-(3-dimethylaminopropyl)carbodiimide and 27 mg of N-hydroxysulfosuccinimide were dissolved in 2 mL of 0.5 M 2-morpholinoethanesulfonic acid (MES) buffer (pH 6.3) in the dark. A 0.2 mL aliquot of the resultant solution was mixed with 0.2 mL of a test MNP (10 mg/mL, in MES) at 25° C. and sonicated for 30 min in the dark, so as to activate the carboxyl groups on the outer surface of the test MNP. The activated MNP thus obtained was magnetically separated from the solution, washed with 0.8 mL of 0.1 M MES, magnetically separated from the solution again, and then suspended in 0.2 mL MED, followed by admixing with 0.1 mL of epirubicin (5 mg/mL, in H2O) by vortexing at 15° C. for 3 hrs. The resultant epirubicin-MNP composite was magnetically separated from the solution and washed with deionized water. The quantity of unbound epirubicin remaining in the composite-free solution was analyzed by HPLC (L-2400 UV-detector and L-2130 pump; Hitachi) using a mobile phase of acetonitrile/methanol/deionized water (15:35:50) at a flow rate of 1.5 mL/min and a measuring wavelength of 256 nm, so that the quantity of epirubicin bound to the test MNP was quantified. The experimental results thus obtained were expressed as mean±SD (n=6). Epirubicin, the test MNP, and the epirubicin-MNP composite thus obtained were analyzed by FT-IR spectroscopy. The epirubicin-MNP-3 composite was further analyzed by confocal microscopy using a TCS SP2 confocal spectral microscope (Leica).

### Results:

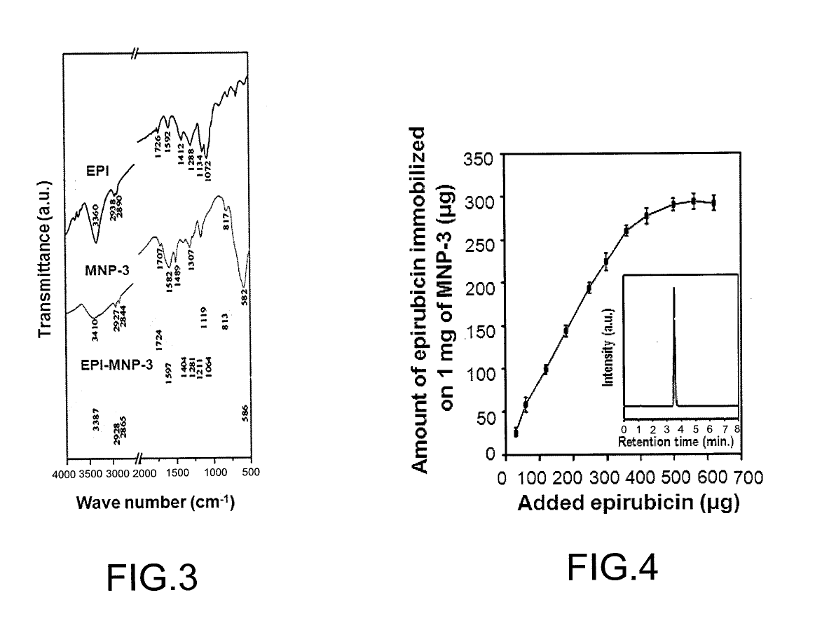

Epirubicin is a cytotoxic anticancer agent used to treat malignant tumors. It is similar in structure to doxorubicin except for a hydroxyl group at the 4′-position of the daunosamine sugar, but has less myocardial and nerve cell toxicity than doxorubicin. Since epirubicin is able to emit orange red fluorescence, the presence thereof can be verified by detecting said fluorescence. When taking the epirubicin-MNP-3 composite as an example, the immobilization of the drug on the surface of MNP-3 was confirmed by the observed orange red fluorescence by confocal microscopy (FIG. 2). Further, it can be seen from FIG. 3 that the epirubicin-MNP-3 composite exhibits four peaks characteristic of epirubicin (1,724 cm⁻¹, 1,404 cm⁻¹, 1,119 cm⁻¹, 1,064 cm⁻¹), indicating that epirubicin was immobilized on the surface of the MNP-3.

MNPs can become saturated with epirubicin because they have a fixed number of carboxyl groups on their surfaces. To decrease the quantity of MNPs required for effective treatment, it is required that the amount of epirubicin immobilized on the surfaces of MNPs reaches a therapeutically effective level as high as possible. FIG. 4 shows the HPLC quantification of epirubicin immobilized on 1 mg of MNP-3 versus added epirubicin, in which the experimental data were expressed as mean±SD (n=6). It can be found that epirubicin immobilization maximized at 300.294 μg of epirubicin bound per 1 mg of MNP-3, equivalent to 452 μg of epirubicin per 1 mg of iron ion.

### 5. Cell Toxicity of Epirubicin-MNP Composite In Vitro

Rat glioma C6 cells were cultured in the culture medium RPMI 1640 supplemented with 2.2 mg/mL sodium carbonate, 10% FBS, 50 μg/mL gentamycin, 50 μg/mL penicillin and 50 μg/mL streptomycin at 37° C. and 5% CO₂. Approximately 10,000 cells were placed in each well of a 96-well culture plate and incubated in a humidified chamber at 37° C. and 5% CO₂ for 24 hrs. Fifty microliters of different concentrations of MNP-3 in medium were then added into the wells, and the cells were cultured for further 24 hrs, followed by the XTT assay to examine cell growth. In another series of experiments, 50 μL aliquots of different concentrations of epirubicin and epirubicin-MNP-3 in medium were added into the wells and the cells were cultured for further 12 hrs, in the absence or presence of an 800-Gauss magnetic field applied beneath the culture plate, followed by the XTT assay to examine cell growth.

Before counting viable cells by the XTT assay, the culture medium in each well was removed and cells were incubated in 120 μL of XTT for 3 hrs. After the reaction, a 100 μL aliquot of the solution in each well was sampled and transferred to a 96-well counting dish, followed by measuring absorbance at OD₄₉₀ using an ELISA reader, so as to determine cell viability (%). The experimental results were expressed as mean±SD. The in vitro cytotoxicities of MNPs, epirubicin and epirubicin-MNPs on C6 cells were evaluated by the IC₅₀ values thereof.

To determine the dose-dependence of epirubicin-MNP toxicity, 2 mL of C6 cells (10,000 cells/mL) were plated in 35-mm diameter plates and cultured in a humidified chamber at 37° C. and 5% CO₂ for 48 h. One hundred microliters of MNPs and different concentrations of epirubicin-MNPs in RPMI medium 1640 were added and the cell cultivation continued for 12 hrs. The medium was removed, cells were washed with 1 mL HBSS and 1 mL Live reagent (Invitrogen) was added. After 30 min, the reagent was removed and the cells were washed again with HBSS. Cytotoxicity was monitored using a TCS SP2 confocal spectral microscope (Leica).

To determine the intracellular distribution of magnetically targeted epirubicin-MNPs, C6 cells were incubated with epirubicin, epirubicin-MNPs, and epirubicin-MNPs in the presence of an 800-Gauss magnetic field applied beneath the culture plate for 4 hrs at 37° C. After washing three times with PBS solution, cells were fixed with 3% glutaraldehyde for 2 h at 4° C., post-fixed with 1% OsO₄ for 1 h at 4° C., washed three times with 0.1 M cacodylate buffer (pH 7.4), and dehydrated using a graded series of ethanol and embedding medium. Cells were embedded in molds in Spurr resin (i.e., 1:1 alcohol:Epon, vol/vol) and polymerized at 60° C. for 24 hrs. Ultrathin sections (80 nm) were cut using a diamond knife and stained with 4% uranyl acetate and lead citrate for 2 hrs and 10 min, respectively. Images were acquired using an H-7500 transmission electron microscope (Hitachi) operating at an acceleration voltage of 100 kV.

### Results:

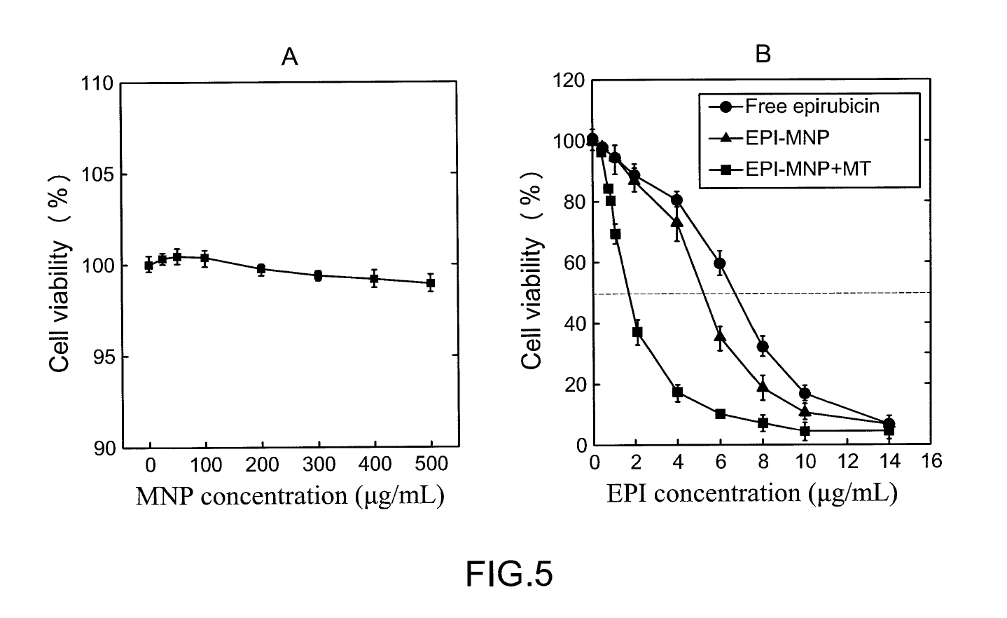

Referring to panel A of FIG. 5, drug-free MNPs had no apparent cytotoxic effect upon cultured rat glioma C6 cells. In contrast, the particles of epirubicin-MNPs composite, presumably taken up by endocytosis, were visible within the cells by TEM, and the particles passed into the nuclei and appeared to have induced apoptosis (data not shown). The number of live cells decreased as the dose of epirubicin-MNPs increased, and tumor cell toxicity was concentrated at the site where the magnet was positioned (data not shown). Conjugating epirubicin to MNPs did not affect the drug's cytotoxicity: the IC₅₀ values of free epirubicin and epirubicin-MNPs were 6.7 μg/mL and 5.2 μg/mL, respectively. The IC₅₀ value of epirubicin-MNPs was reduced significantly to 1.7 μg/mL when magnetic targeting (MT) was applied (FIG. 5, panel B).

6. Effect of Focused Ultrasound (FUS) and Magnetic Targeting (Mt) Upon Delivery of MNPs into Brain

This experiment was conducted to examine the effect of FUS and MT, either singly or in combination, upon delivery of MNPs to animal brain.

### Animal Preparation.

All animal experiments were approved by the Institutional Animal Care and Use Committee of Chang Gung University and adhered to their experimental animal care guidelines. Normal Sprague-Dawley rats (300-400 g in weight) were purchased from BioLasco Taiwan Co., Ltd. Thirty-nine rats were tested to confirm the efficacy of the proposed approach. Brain tumors were induced in another 14 rats by injection of cultured C6 tumor cells. Briefly, C6 tumor cells (10⁶ cells/plate) were injected into the brains using a microdialysis pump system (CMA Microdialysis). Animals underwent FUS treatment on Day 10 after tumor implantation to determine if the method could open an area of the BBB sufficiently large to cover the area of the induced tumors.

### Focus Ultrasound (FUS) Sonication

Before the FUS treatment, the rats were anesthetized by i.p. injection of chlorohydrate (30 mg/kg). The top of the cranium of each rat was shaved with clippers, and a PE-50 catheter was inserted into the jugular vein. The animal's head was attached tightly to a 4-cm² thin-film window directly under an acrylic water tank. The animal's cranial opening was filled with degassed water to serve as an acoustic coupling device. SonoVue® SF6-coated US microbubbles (mean diameter 2-5 μm, 2.5 μg/kg; Bracco) were administered i.v. before sonication (with a time lapse<10 s). Each bolus injection contained 0.1 mL of microbubble/kg of body weight mixed with 0.2 mL of saline solution. A heparin flush (0.2 mL) was subsequently performed.

Animal experiments were monitored using the 3-T magnetic resonance imager to localize the geometric center of the FUS and the energy exposure site. US was delivered to the brain transcranially using a MR-compatible spherical transducer (diameter, 60 mm; radius of curvature, 80 mm; frequency, 400 kHz, electric-to-acoustic efficiency, 70%; Imasonics) with the center of the focal zone positioned at a penetration depth of 2 to 3 mm in each hemisphere. Single burst-mode US was delivered, with a burst length of 10 ms, a pulse-repetition frequency of 1 Hz, and total sonication duration of 120 s. For optimization studies, the input electric power used was 2 W, corresponding to an acoustic negative peak (i/e., spatial-peak, temporal-peak) pressure amplitude measured through the animal cranium equal to 0.62 MPa.

### FUS Calibration and Treatment

To measure the calibrated US pressure and the focal beam dimensions, a polyvinylidene difluoride hydrophone (0.5-mm diameter; 50 kHz to 20 MHz calibration range; Onda) was mounted on an in-house-designed 3D positioning system. Measurements were conducted in a tank filled with degassed water. To estimate peak pressures in vivo, measurements were conducted with and without harvested rat skull samples: the mean energy loss caused by the skull was calculated to be 77%. The pressure was reduced further by 5 Np/m/MHz to account for US attenuation by the brain itself. The half-maximum pressure amplitude diameter and the length of the produced focal spot were determined to be approximately 4 mm and 23 mm, respectively.

### Magnetic Targeting (MT) Treatment

In the MT treatment, a permanent magnet having a peak magnetic flux density of 0.18 T, 0.4 T or 0.55 T was used to produce an inhomogeneous magnetic field. To concentrate the magnetic flux density onto the disrupted region of the BBB, the magnet was tilted at an angle relative to the animal's brain, attached to the animal's scalp, and supported and tightened using a custom-made plastic belt for the desired duration (3-24 hrs). MRI images were acquired immediately after removal of the magnet.

### Magnetic Resonance Imaging (MRI) Monitoring

Animals' brains were monitored by MRI after the FUS treatment and/or the MT treatment. All MRI images were acquired on a 3-T scanner using the standard wrist coil with an inner diameter of 13 cm. The animals were anesthetized with 2% isoflurane throughout the MRI imaging process, placed in an acrylic holder, and positioned in the center of the magnet. An i.v. bolus (0.1 mmol/kg) of gadopentetate dimeglumine MRI contrast agent (Magnevist; Berlex) was administered before scanning. To identify the region of the BBB disrupted by the FUS treatment, contrast-enhanced T1 turbo spin-echo sequences were acquired using the following parameters: repetition/echo time, 780 ms/15 ms; slice thickness, 1.4 mm; matrix size, 128×256; field of view, 39×60 mm (resolution, 0.3×0.3 mm). T2-weighted images were obtained to produce R2 maps both in gel phantom or in vivo experiments by using a double-TE spinecho sequence and acquired three times, using the following parameters: repetition time, 3,860 ms, echo time, 8/14, 28/57, and 85/228 ms; matrix, 128×256; field of view, 38×76 mm (resolution, 0.3×0.3 mm); and slice thickness, 1.4 mm. T2*-weighted images were used to observe the distribution of MNPs 3, 6, 12, and 24 hrs after MNP injection. T2-weighted imaging was also used to measure tumor volume.

### Measurement of MNP and Epirubicin Deposition in Tissue

Animals were killed at 3, 6, 12, and 24 hr after MNP injection. Brains were collected immediately, washed twice with normal saline solution, and dried under vacuum for 48 hrs at 80° C. The dried samples were ground into powder, and the powders were acid-digested in 12 M aqua regia overnight. The iron content of the samples was measured by ICP-OES; each assay was performed in triplicate. Epirubicin concentration was calibrated by HPLC using a L-2400 UV detector and L-2130 pump (Hitachi) and a Supelcosil LC-18 column (4.6×250 mm). Each assay was performed in triplicate. The epirubicin concentration measured in tissues was 383 to 415 μg epirubicin/mg iron ion, with a consistent mean immobilization ratio of 395.6±17.3 μg epirubicin/mg iron ion (data not shown). Thus, epirubicin concentration in tissues can be quantified reliably by measuring MNP concentration.

### Epirubicin Concentration in Brain Tissue

Animals were killed 3, 6, 12, and 24 hr after treatment. Brain tissues were transferred to a 2-mL microtube and epirubicin was extracted with 2N hydrochloric acid by shaking for 60 min at 10° C. After centrifugation, the supernatant was filtered (0.22 μm) and each extract was analyzed by HPLC and ICP-OES. The mobile phase for HPLC was acetonitrile:methanol:deionized water (15:35:50, vol/vol/vol) with 0.5 mL phosphoric acid (85%) per 500 mL mobile phase pumped at a flow rate of 1.5 mL/min and a measuring wavelength of 256 nm. Tissue concentrations were determined from an epirubicin standard curve (1-40 μg/mL; retention time, 3.89 min).

### Histology and Microscopy

Evans blue was administered after the FUS treatment to confirm BBB disruption. Brain tissues were prepared after in vivo MRI analysis. Animals were killed 3, 6, 12, and 24 h after injection of dye and/or MNPs. Slides were stained with Prussian blue (Sigma) to detect iron deposited in cells/tissue samples. Briefly, brain sections mounted on slides were stained in a 1:1 mixture of 2% potassium ferrocyanide and 2% hydrochloric acid for 30 min at room temperature. The slides were rinsed with distilled water, counterstained with Nuclear Fast Red for 5 min, dehydrated, and photographed. Nuclei were stained with the fluorescent dye DAPI. Microscopic observations were performed using a Zeiss Axioplan imaging 2 microscope with AxioVision 4.1 imaging software, an AxioCam HRc camera, and Fluar 10×/0.50, Plan-Apochrome 20×10.75, and Plan-Neofluar 100×/1.30 oil objectives (Carl Zeiss).

### Results:

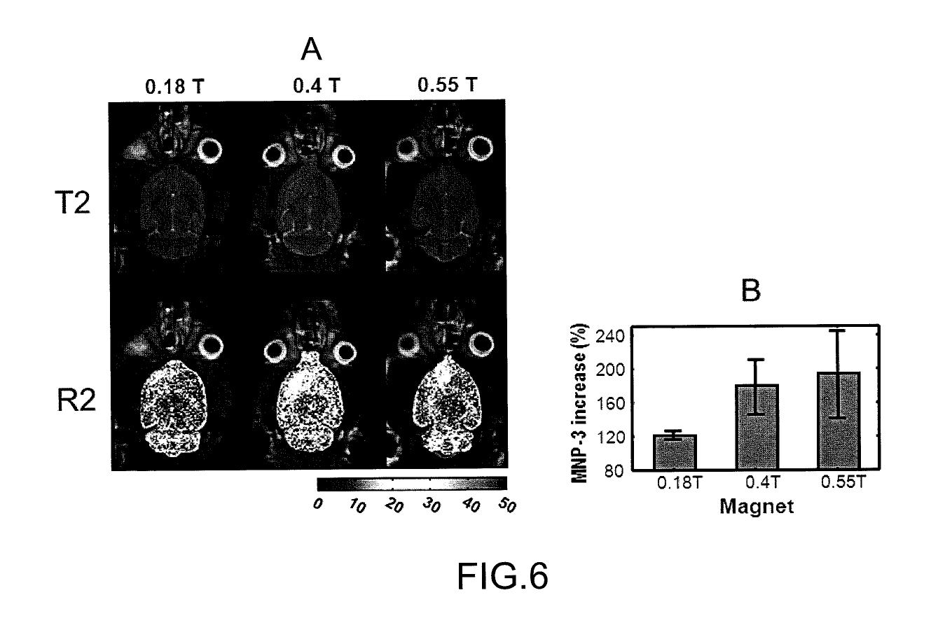

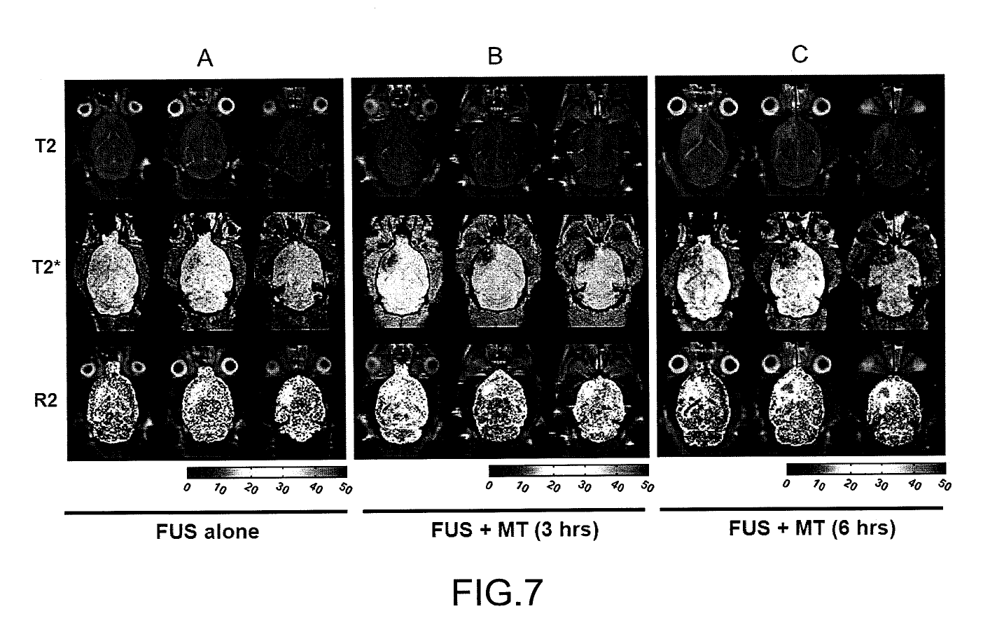

The enhancement of local MNP delivery into brain via combined FUS and MT treatment was evaluated. MNP deposition in the brain was confirmed histologically by Prussian blue staining of iron deposits (data not shown). Contrast-enhanced T1-weighted images confirmed that the FUS treatment disrupted the BBB (data not shown). Local MNP capture was dependent on field strength: a 0.55-T magnet attracted four times more MNPs than a 0.18-T magnet, as assessed by R2 mapping (FIG. 6, panels A and B). R2 maps (showing changes caused by different amounts of MNP) and T2* imaging (indicating susceptibility artifact-induced signal loss caused by MNP accumulation) showed that the FUS treatment alone increased local deposition of MNP-3 by 21.5% relative to the contralateral hemisphere (FIG. 7, panel A). Subsequently applying MT increased MNP accumulation (FIG. 7, panels B and C), with a 6-h exposure caused the greatest increase in MNP concentration (244.6% relative to the contralateral hemisphere) but also a wider distribution (as seen in T2* imaging) relative to brains treated with FUS alone (FIG. 7, panel C). The combination of the FUS treatment and a 6 hr MT treatment deposited 21,738±3,477 ng of epirubicin per gram of brain tissue, whereas treatment with the FUS treatment alone accumulated only 1,336±1,182 ng epirubicin per gram of tissue.

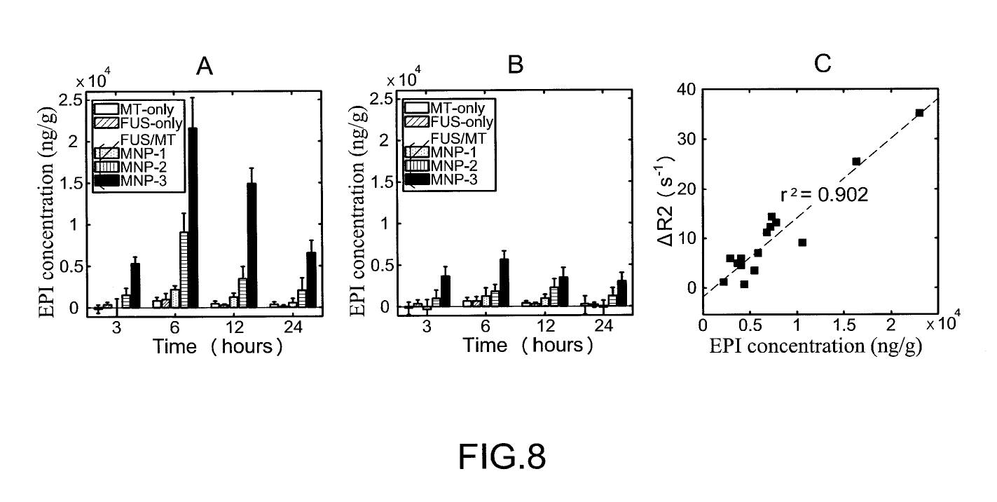

The amounts of epirubicin-MNPs accumulated in brain parenchyma were also measured by inductively coupled plasma optical emission spectrometry (ICP-OES) and the epirubicin concentration was measured by HPLC (FIG. 8). Neither FUS alone nor MT alone caused significant MNP accumulation in the experimental brain site (FIG. 8, panel A). Furthermore, accumulation was no more significant on the experimental side relative to the contralateral side (FIG. 8, panel B) with either of these treatments. However, when FUS and MT were combined, MNP accumulation increased dramatically, with epirubicin-MNP-3 showing the highest levels of accumulation.

Furthermore, the accumulation was markedly more pronounced on the side subjected to the MT treatment. Interestingly, when epirubicin-MNP-2 was used, the ratio of MNP accumulation between the experimental and contralateral hemispheres reached a maximum at 6 hr but decreased quickly at 12 hr of MT, whereas the ratio was maintained when epirubicin-MNP-3 was administered. This suggests that MNPs with higher R2 values can contribute a higher magnetic moment (thus enhancing MT and maintaining localized epirubicin concentrations at therapeutic levels) and are less likely to be cleared from the bloodstream as foreign bodies than those with lower R2 values. Most importantly, this ratio correlated highly (r²=0.908) with the values measured in vivo using R2 maps, indicating that such maps provide a good estimation of MNP (and thus drug) localization and concentration in vivo (FIG. 8, panel C). Based on comparisons between the ICP-OES/HPLC analyses and the R2 relaxivities determined by MRI, 1 mM (or 4×10⁻³ mmol) of MNP-3 detected on R2 maps was equivalent to an epirubicin concentration of 133,894 ng/g of tissue, or 617 ng/g of epirubicin per change in R2 (in s⁻¹).

### 7. Enhancement of Therapeutic MNP Delivery to Brain Tumors

This experiment was performed to investigate the effect of FUS and MT upon delivery of a drug-MNP composite to a brain tumor.

### Analysis of Tumor Progression and Animal Survival

Animals with induced tumors were used to analyze survival. As a control group, rats (n=12) were injected with C6 glioma cells but received no further treatment; rats in groups 2 (n=9), 3 (n=6), and 4 (n=11) all received a single dose of therapeutic MNPs (4 mg/kg) administered i.v. at Day 10 after tumor cell implantation. Group 3 rats were then subjected to FUS sonication Day 10 after implantation, whereas group 4 rats were subjected to both FUS sonication and a 6 hr MT treatment on Day 10. The statistical significance of the increase in tumor volume increase was determined using a two-tailed unpaired t test, with P<0.05 considered to be significant. The Kaplan-Meier method was used to plot animal survival. Statistical significance was calculated using the Mantel-Cox test, and the tests were considered significant at P<0.05. The different treatment groups were compared in terms of median survival time, percent increase in median survival time, and maximal survival.

### Results:

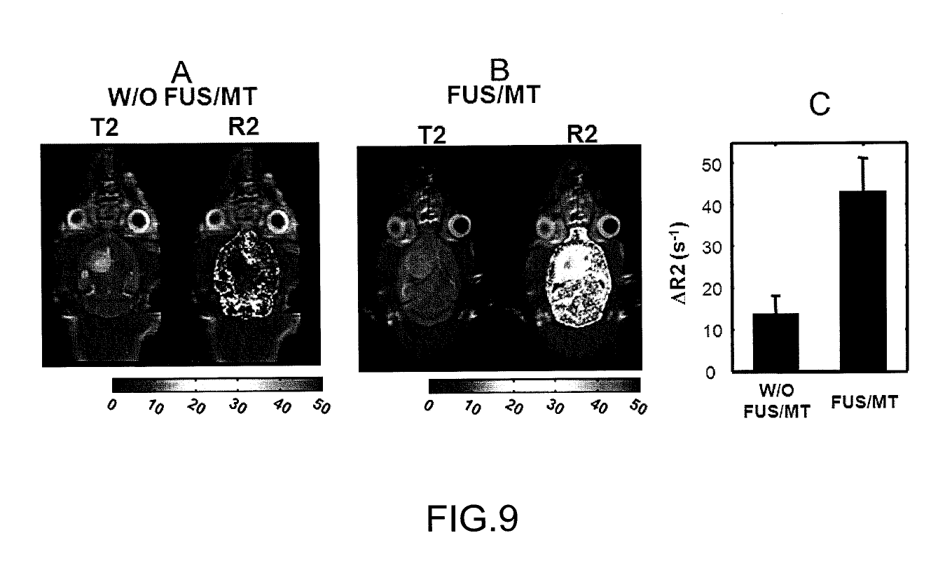

Tumor bearing animals were treated with epirubicin-MNP without (control) or with combined FUS/MT treatment. Control animals showed no MNP accumulation in the tumor region 6 hr after epirubicin-MNP administration (FIG. 9, panel A). However, relaxation rates increased 2.6-fold (to 35.8±5.2 s⁻¹ relative to control values of 13.6±4.5 s⁻¹) at the tumor site by applying MT for 6 h after FUS treatment (FIG. 9, panels B and C). After correcting for the baseline value contributed by blood circulation, it was estimated that 0.16±0.03 mM of MNPs was delivered to the tumor, equivalent to 11,982±2,105 ng of epirubicin per gram of tissue. This is approximately 15-fold higher than the therapeutic range (819±482 ng/g tumor) reported for in vivo doxorubicin, which has a clinical response rate of 39% in patients with breast carcinoma (Treat L H, et al., (2007), Int. J. Cancer, 121:901-907).

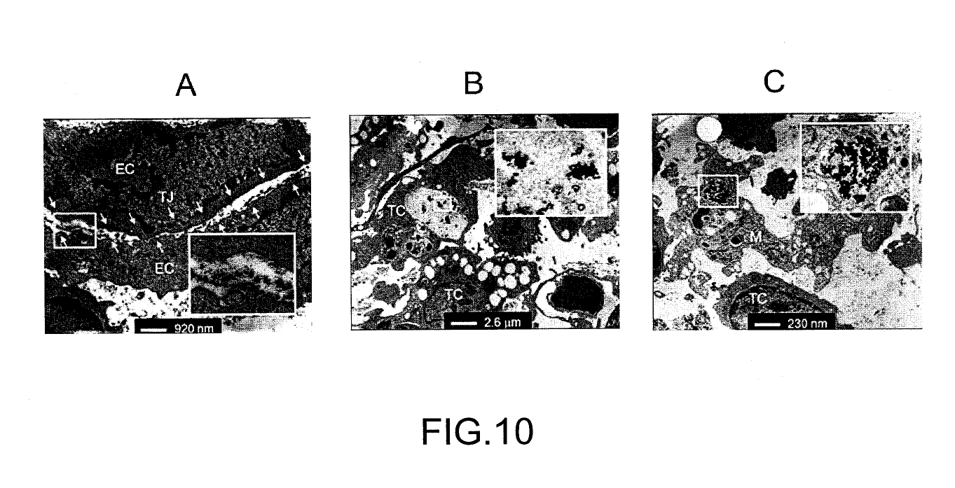

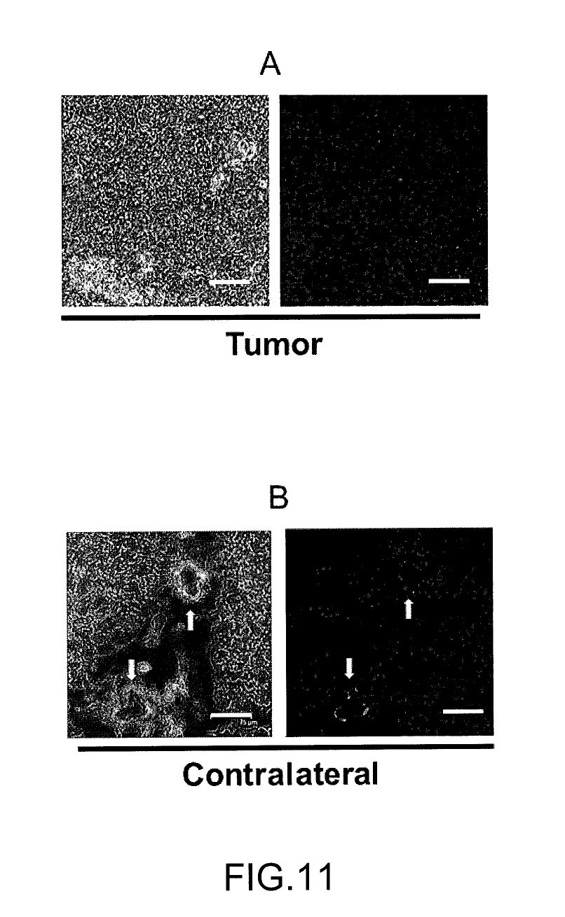

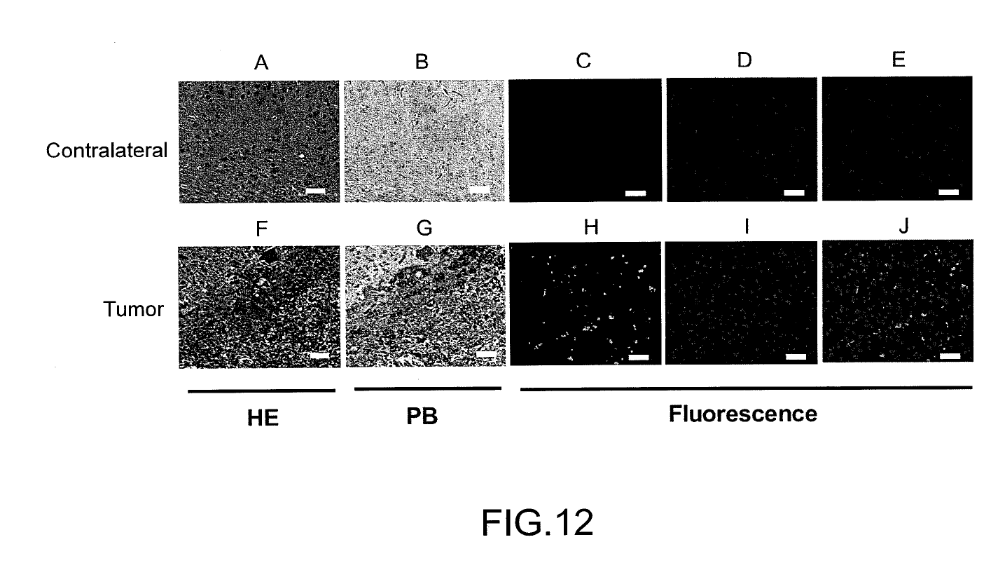

Furthermore, TEM showed that FUS apparently induced interendothelial clefts with no obvious tight-junctional complexes in tumors (FIG. 10, panel A), and that epirubicin-MNPs were taken up by tumor cells and macrophages (FIG. 10, panels B and C). In contrast, MNP injection alone caused no ischemic or histological changes in the brain during the period studied. Confocal and fluorescence microscopy (FIG. 11, panel A and B; and FIG. 12, panels C and H) and Prussian blue staining (FIG. 12, panels B and G) confirmed that more epirubicin-MNPs were deposited at the tumor site than in the contralateral side. Thus, epirubicin localization was enhanced significantly within the brain parenchyma by FUS/MT, whereas it appeared in only the capillary bed in the contralateral hemisphere, indicating that off-target effects were minimal. Furthermore, the correlation between MNP distribution (as determined by Prussian blue staining) and epirubicin fluorescence confirmed that MNPs are effective carriers for epirubicin, and by extension, that MRI R2 mapping can be used to detect localized concentrations of the drug with a high degree of precision.

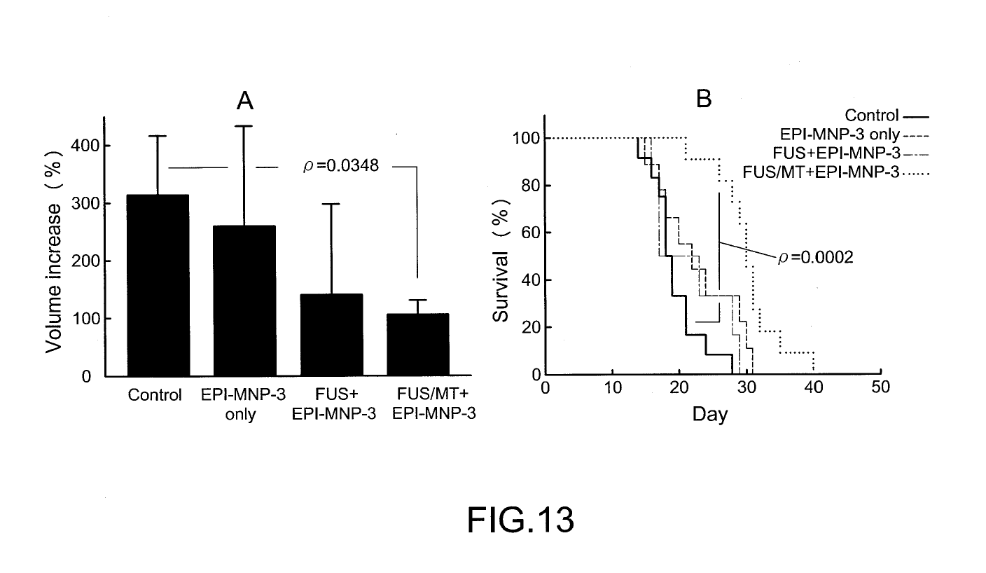

Experimental treatment of animals with induced tumors showed that combining therapeutic MNPs with FUS/MT provided the most effective means of controlling tumor progression: over a 7-day period, tumor volume increased only 106±24% in treated animals, as compared to a 313% increase (±103%) in controls. Furthermore, although treatment with epirubicin-MNPs alone or epirubicin-MNPs plus FUS improved median animal survival to only 23 days and 20 days, respectively, survival improved significantly in animals receiving epirubicin-MNPs under the FUS/MT treatment (median survival, 30.5 days vs. 18.3 days, or a 66% improvement over control; P=0.0002; FIG. 13).

### Discussion

### Delivery of Macromolecular Therapeutic Agents to the CNS

FUS can temporarily disrupt the BBB, increasing local EPR in the CNS. This technology is ideally suited for transcranial delivery of drugs with molecular weights greater than 400 Da (W. M. Pardridge (2002), supra; L. L. Muldoon et al. (2007), J. Clin. Oncol., 25:2295-2305). However, although this technique works with substances with molecular weights as high as 150 kDa, penetration is still hampered at molecular weights of 2,000 kDa (approximately equivalent to 55 nm, as measured by TEM (J. J. Choi et al. (2010), Ultrasound Med. Biol., 36:58-67).

The current strategy to assess delivery of therapeutic substances (e.g., 50-150 kDa monoclonal antibodies or <1 kDa chemotherapeutic agents), involves their co-administration with a separate gadolinium type T1 contrast agent (<1 kDa). However, this technique estimates drug concentration indirectly, assuming a correlation between changes in image contrast and the concentration of the delivered substance. Also, conjugating small contrast agents (e.g., gadolinium) with therapeutic substances does not permit active targeting. However, an imaging probe that also has a therapeutic effect and/or target specificity must bind the agent to be delivered to the contrast agent, increasing the compound size and thus decreasing the likelihood that FUS will stimulate EPR.

The study of this invention confirms that combining passive and active transport mechanisms can deliver large multifunctional molecules to the CNS. FUS treatment has been used safely to deliver 886±327 ng doxorubicin per gram of tissue into normal brains (L. H. Treat et al., (2007), Int. J. Cancer, 121:901-907). This is comparable to the levels of epirubicin delivered by FUS treatment alone in the study of this invention (1,197±226 ng/g and 1,162±1,028 ng/g, as assessed by MRI and ICP-OES/HPLC detection, respectively). Doxorubicin delivery could be increased to 5,336±659 ng/g (L. H. Treat et al. (2007), supra), but at the expense of increased damage to brain tissue. In contrast, the approach developed in this invention synergistically combines FUS and MT to increase epirubicin delivery to the brain tumors by at least an order of magnitude (21,738±3,477 ng/g and 22,070±3,205 ng/g, as assessed by MRI and ICP-OES/HPLC detection, respectively) using a “safe” level of ultrasound exposure.

### Use of R2 Maps and T2* Images to Detect MNPs

The study of this invention used T2*-weighted images and quantitative R2 maps to detect MNP accumulation in the brain in vivo. The T2*-weighted images showed increased sensitivity to the local field inhomogeneity induced by MNPs. Nonhomogeneous distribution or local accumulation of MNPs leads to an additional loss of phase coherence (i.e., dephasing) of the spins. This decreases the transverse relaxation times and thus contributes to a reduction in signal intensity (C. Corot et al., (2006), Adv. Drug Deliv. Rev., 58:1471-1504), allowing such images to be used as a direct indicator of MNP distribution. It should be noted, however, that, although conceptually feasible, quantification of R2* (i.e., 1/T2*) is potentially non-reproducible because iron deposited over multiple sessions can produce strong magnetic field susceptibility, resulting in differences in field inhomogeneity (H. Dahnke et al. (2005), Magn. Reson. Med., 53:1202-1206). In contrast, R2 maps showed the high spin-spin relaxivity of MNPs, which was linear. The disadvantage of this approach is that multiple T2-weighted acquisitions at different echo times are required and comprehensive post-processing is necessary. Nevertheless, different information can be extracted from these two methods. For example, enhancements seen in T2*-weighted imaging contain higher spatial resolution and can show local concentration/aggregation of MNPs, whereas R2 maps provide a rather averaged MNP amount per unit volume. Thus, combining these techniques provides image resolution and quantitative information on MNP depositions.

### Enhanced Drug Delivery to the BBB-Intact CNS

Most drugs used to treat CNS diseases that do not compromise the BBB (e.g., neurodegenerative diseases) must have sufficiently small molecular weights and need to be uncharged (or only partially ionized) at physiological pH to allow passive diffusion into the brain. Increasing the lipid solubility of a drug to enhance BBB penetration can have undesirable effects such as decreasing overall solubility and/or bioavailability, increasing plasma protein binding, and increasing uptake by the liver or reticuloendothelial system.

Instead of designing therapeutic agents sufficiently small to penetrate the BBB, another approach is to temporarily open the BBB. Osmotically opening the barrier by infusing hypertonic mannitol through the carotid artery has been used successfully to treat human brain tumors (S. I. Rapoport et al. (2000), Cell Mol. Neurobiol., 20:217-230; E. A. Neuwelt et al. (1982), Proc. Natl. Acad. Sci., USA, 79:4420-4423). Alternatively, bradykinin acting via B2 receptors in the luminal membrane of the endothelium can permeabilize the BBB, presumably by elevating intracellular freecalcium levels and thus modulating tight junctions (D. F. Emerich et al. (2001), Clin. Pharmacokinet., 40:105-123). Alkylglycerols administered via the carotid artery can also modulate the BBB (H. J. Lee et al. (2002), J. Drug Target., 10:463-467). However, none of these approaches open the BBB locally, and this lack of selectivity can cause undesirable side effects in normal portions of the brain, as well as presenting systemic hazards (K. Hynynen et al. (2006), supra). The use of noninvasive FUS to temporarily disrupt the BBB locally provides an ideal solution to the problems of drug design and localized delivery of such drugs to deep CNS tissues.

Enhanced Drug Delivery into Brain Tumors

Primary brain tumors or metastases to the brain from breast or lung cancers have markedly different vascular and hemodynamic characteristics than the intact brain (D. R. Groothuis et al. (1982), J. Neuropatho. Exp. Neurol., 41:164-185; M. S. Lesniak and H. Brem (2004), Nat. Rev. Drug Discov., 3:499-508; E. A. Neuwelt et al. (1985), Cancer Res., 45:2827-2833; E. A. Neuwelt et al. (1986), Cancer, 58:1609-1620). Vascular characteristics in tumors vary widely, and permeability does not necessarily correlate with tumor histology, size, or anatomical location. All these can restrict chemotherapeutic agents from reaching a therapeutic dose (O. Arosarena et al. (1994), Brain Res., 640:98-104; R. G. Blasberg et al. (1983), J. Neurosurg., 58: 863-873; A. P. Pathak et al., (2001), Magn. Reson. Med., 46:735-747). Even small lipid-soluble agents such as 1-3-bis(2-chloroethyl)-1-nitrosourea (BCNU), which in its natural form can penetrate the BBB, cannot accumulate in tumors (H.-L. Liu et al. (2010), Radiology 255:415-425). Low permeability and retention are even more pronounced in the case of large chemotherapeutic agents that cannot penetrate the BBB, such as lipid-encapsulated doxorubicin (L. H. Treat et al. (2007), supra) or the epirubicin-MNPs used in the study of this invention. In an earlier study, the applicants found that the combined use of FUS and MT improved the delivery of BCNU to rodent gliomas (Pin-Yuan Chen et al. (2010), “Novel magnetic/ultrasound focusing system enhances nanoparticle drug delivery for glioma treatment,” Neuro-Oncology, doi:10.1093/neuonc/noq054, Epub 2010 Jul. 27). The experimental results obtained herein confirm that, by enhancing BBB permeability in tumors with FUS and concurrently applying MT, one can successfully deliver multifunctional macromolecules not only into brains with intact BBBs, but also to those in which the barrier has been compromised by pathological conditions, expanding the range of such molecules that could be used for integrated diagnostic and therapeutic treatments. Furthermore, the efficacy of such a treatment regimen has been confirmed experimentally (FIG. 13).

### Potential Parameters for MT

Optimizing the specifications for MT can maximize active MNP delivery. The particles must be of a size sufficient to generate a strong enough magnetic moment such that, when the tumor (or the vascular system surrounding the tumor) is exposed to the magnetic field, the net attractive force acting on the MNPs can overcome the viscous drag and allow the particles to reach the targeted area. Active targeting can occur only when the MNPs are magnetized, and the efficiency of such magnetization varies with their crystalline structure, the process used to synthesize them, the material with which they are coated, and other factors, particularly size (S. Goodwin et al. (1999), J. Magn. Magn. Mater., 194:132-139). For example, larger MNPs can induce thrombus in blood vessels more easily, whereas small MNPs (<100 nm) can be difficult to attract with low-strength magnetic fields, making size selection a dilemma. Previous studies showed that when a relatively strong magnetic field (>0.4 T) was applied, MNPs having high relaxivity (>40 mM⁻¹s⁻¹) could be used successfully for MT to superficial tissues (B. Chertok et al. (2007), J. Control. Release, 122: 315-32333; B. Chertok et al. (2008), Biomaterials, 29:487-496).

Although the present study demonstrates that MT can be used to deliver therapeutic drugs in small animal brains, it should be noted that the distance between the magnet pole and the animal brain was still short (<10 mm). Scaling up the current setup for clinical applications will still be challenging: The magnetic flux density from the single-pole magnet decayed sharply and was unable to provide effective MT in deep-seated tissues (data not shown). Possible improvements include increasing the magnet flux density by replacing the current permanent magnet with a superconducting electrical magnet coil, modifying the open-pole structure to a two-pole or closed-loop frame design (to reduce magnetic flux drop) (P. Dames et al. (2007), Nat. Nanotechnol., 2:495-499), or designing MNPs with the use of highly magnetic materials (H. Lee et al. (2009), Proc. Natl. Acad. Sci., USA, 106: 2459-12464).

In conclusion, this invention provides an integrated nanomedicine platform to enhance and monitor delivery of multifunctional nanoparticles to the brain. FUS can locally and transiently disrupt the BBB, and the subsequent application of MT significantly improves deposition of therapeutic MNPs (at least an order of magnitude higher than previously reported approaches). The combined use of these techniques provides an important means to deliver therapeutic doses locally and simultaneously reduces the problem of systemic toxicity common to i.v.-administered therapeutic agents. More importantly, MNP distribution can be monitored by MRI, permitting quantification of drug delivery in real time in vivo.

All patents and literature references cited in the present specification as well as the references described therein, are hereby incorporated by reference in their entirety. In case of conflict, the present description, including definitions, will prevail.

While the invention has been described with reference to the above specific embodiments, it is apparent that numerous modifications and variations can be made without departing from the scope and spirit of this invention. It is therefore intended that this invention be limited only as indicated by the appended claim.

We claim:

1. A method for treating a brain disease, comprising:
(i) delivering therapeutic agent-magnetic nanoparticle composites to the vicinity of the blood-brain barrier of a subject in need of such treatment;
(ii) applying focused ultrasound to the subject so as to open the blood-brain barrier of the subject;
(iii) applying a magnetic field to the subject to direct the therapeutic agent-magnetic nanoparticle composites across the blood-brain barrier to a designated locus inside the brain of the subject;
(iv) monitoring the quantity of the therapeutic agent-magnetic nanoparticle composites present at the designated locus by magnetic resonance imaging;
and optionally, directing more therapeutic agent-magnetic nanoparticle composites to the designated locus inside the brain of the subject by repeating steps (i) to (iv),
wherein each of the therapeutic agent-magnetic nanoparticle composites is constructed to comprise:
(a) a magnetic nanoparticle formed of an iron-based core and a shell encapsulating the iron-based core, the shell comprising a biological compatible polymer, and
(b) a therapeutic agent bound to the shell of the magnetic nanoparticle;
wherein the magnetic nanoparticle has an average particle size ranging from 5 to 200 nm and wherein reaction conditions used in preparation of the iron-based core of the magnetic nanoparticle are controlled, so that the iron-based core has a crystalline structure that imparts the magnetic nanoparticle composites with a sufficiently high magnetization, thereby enhancing magnetic targeting of the therapeutic agent-magnetic nanoparticle composites to the designated locus inside the brain of the subject; and
wherein in step (iii), the magnetic field is generated by a magnet that provides a magnetic flux density not less than 0.18 T.

2. The method of claim 1, wherein step (ii) is performed by applying to the subject a planar/focused ultrasound beam having a frequency ranging from 20 kHz to 10 MHz, at a sonication duration ranging from 100 nanoseconds to 30 minutes, with continuous wave or burst mode operation, in which frequency of burst mode repetition varies from 0.01 Hz to 1 MHz.

3. The method of claim 1, wherein prior to step (ii), the subject is administered with ultrasound microbubbles that enhance focused ultrasound.

4. The method of claim 1, wherein in step (iii), the magnetic field is generated by a magnet that provides a magnetic flux density ranging from 0.18 T to 0.55 T.

5. The method of claim 1, wherein the magnetic nanoparticle has a saturated magnetization ranging from 32.6 emu to 81.7 emu based on one gram of the magnetic nanoparticle.

6. The method of claim 1, wherein the magnetic nanoparticle has a saturated magnetization greater than 70 emu per gram of the magnetic nanoparticle.

7. The method of claim 1, wherein the iron-based core of the magnetic nanoparticle has a relaxivity not less than 30 mM⁻¹s⁻¹.

8. The method of claim 7, wherein the iron-based core of the magnetic nanoparticle has a relaxivity in a range from 30 to 400 mM⁻¹s⁻¹.

9. The method of claim 1, wherein the iron-based core of the magnetic nanoparticle is made of a material selected from the group consisting of Fe₂O₃ and Fe₃O₄.

10. The method of claim 1, wherein the iron-based core of the magnetic nanoparticle is made of Fe₃O₄.

11. The method of claim 1, wherein the biological compatible polymer used to form the shell of the magnetic nanoparticle is selected from the group consisting of polyaniline, polylactic acid, polyglycolic acid, polylactic polyglycolic acid, dextran, dextran grafted with poly(N-isopropylacrylamide-co-N,N-dimethylacrylamide), dextran grafted with poly(phosphoester urethane), polycaprolactone, polyhydroxybutyrate, polyethylene glycol-modified polylactic polyglycolic acid, and poly(L-lysine)-g-polyethylene glycol)-modified polylactic polyglycolic acid.

12. The method of claim 11, wherein the biological compatible polymer is carboxy-functionalized polyaniline.

13. The method of claim 12, wherein the biological compatible polymer is poly[aniline-co-N-(1-one-butyric acid)]aniline.

14. The method of claim 1, wherein the brain disease is selected from tumors, cancer, degenerative disorders, sensory and motor abnormalities, seizure, infection, immunologic disorder, mental disorder, behavioral disorder, localized CNS disease, and combinations thereof.

15. The method of claim 1, wherein the therapeutic agent is selected from neuropharmacologic agents, neuroactive peptides, proteins, enzymes, gene therapy agents, neuroprotective or growth factors, biogenic amines, trophic factors to brain or spinal transplants, immunoreactive proteins, receptor binding proteins, radioactive agents, antibodies, and cytotoxins.

16. The method of claim 1, wherein the brain disease is brain cancer.

17. The method of claim 16, wherein the therapeutic agent is an anti-brain cancer drug selected from the group consisting of epirubicin, doxorubicin, 1,3-bis(2-chloroethyl)-1-nitrosourea (BCNU), N(2-chloroethyl)-N′-cyclohexyl-N-nitrosourea (CCNU), methyl 6-(3-(2-chloroethyl)-3-nitrosoureido (MCNU), (2-chloroethyl)nitrosourea (CI-ENU), N-(2-hydroxyethyl)-N-nitrosourea (HO-ENU), and 1-methyl-1-nitrosourea (MNU).
